(19) United States
(12) Patent Application Publication
(10) Pub. No.: US 2026/0309606 A1
(43) Pub. Date: Oct. 8, 2026

(54) METHOD FOR MEASURING DOWNHOLE HOLDUP, AND METHOD FOR DETERMINING DOWNHOLE FLUID INTERFACE AND GEOMETRIC DISTRIBUTION

(71) Applicant: China University of Petroleum (East China), Qingdao (CN)

(72) Inventor: Xuanhua ZHANG, Qingdao (CN)

(21) Appl. No.: 19/682,598

(22) Filed: May 20, 2026

(30) Foreign Application Priority Data
Feb. 28, 2026 (CN) 202610236572.2

(51) Int. Cl. G01N 9/00; E21B 49/08; G01N 33/28

(52) U.S. Cl. CPC G01N 9/002; E21B 49/0875; G01N 33/2823; G01N 33/2847; G01N 2009/006

(57) ABSTRACT

The present disclosure relates to a method for measuring downhole holdup, and a method for determining downhole fluid interface and geometric distribution, which belongs to the field of oil and gas exploration technology. The method for measuring downhole holdup uses a plurality of tuning forks distributed on the flow cross-section to achieve the oil-water determination by comparing the fluid density collected by tuning forks with a preset oil-water density discrimination threshold. The time proportion of oil/water collected by the tuning forks is used to characterize downhole fluid holdup. This method offers advantages such as good versatility, high measurement efficiency, and high resolution. The method for measuring downhole fluid interface uses a plurality of tuning forks distributed on the flow cross-section to achieve the oil-water determination. Based on the oil-water alternating switching phenomenon, oil-water interface points are identified, and the oil-water interface of the horizontal well is determined.

### TECHNICAL FIELD

The present disclosure belongs to the field of oil and gas exploration technology, particularly to a method for measuring downhole holdup, and a method for determining downhole fluid interface and geometric distribution.

### BACKGROUND

In oil and gas wells, the flow in pipes involves different types of light and heavy phases (oil, gas, and water), varying densities and flow rates of each phase, and different well conditions (vertical or horizontal wells), leading to highly complex downhole fluid flow. Due to density differences among fluid phases, a fluid interface phenomenon may appear among the phases in horizontal wells, whereas in vertical wells, different geometric distribution phenomena of the fluid phases also exist on the flow cross-section. Under such complex downhole flow conditions of oil and gas wells, determining the holdup of each phase on the flow cross-section, as well as the fluid interface or geometric distribution of each phase, has long been a challenge.

Since the 1950s and 1960s, downhole fluid holdup measurement techniques both domestically and internationally have been characterized by capacitive water holdup meters, radioactive water holdup meters, and downhole imaging logging technology based on fluid conductivity flow imagers. There have been no significant technological breakthroughs in this field since that period. Among these, the capacitive water holdup meter is the most commonly used and is divided into a continuous capacitive water holdup meter and a sampling capacitive water holdup meter. Both have their advantages and disadvantages, as detailed below: The continuous capacitive water holdup meter enables continuous measurement. However, when the water holdup exceeds 30%, the internal and external electrodes of its capacitor become conductive, and regardless of further changes in water holdup, the instrument response becomes a straight line, and loses resolution. Consequently, the resolution limitation of the continuous capacitive water holdup meter makes it unsuitable for use in the high water cut stages of oilfields. The sampling capacitive water holdup meter performs measurement after sampling and allows the mixture to stand still until the oil and water separate. It offers high resolution and can measure water holdup in high-water-cut wells. However, its sampling and separation test time is long (approximately 30 min per measurement point), resulting in low measurement efficiency. The radioactive water holdup meter requires a radioactive source, which is costly. The flow imager determines oil or water by the difference in conductivity when its probe encounters oil or water. However, its probe is easily wrapped by an oil film, isolating the probe from the test environment, which can cause the imager to misidentify oil when passing through water, greatly reducing resolution.

Moreover, regarding the oil-water flow interface in horizontal wells, the flow patterns and interface of oil-water two-phase can be clearly observed in laboratory horizontal pipe simulations. However, no direct and effective test method exists for determining the downhole oil-water flow interface. Similarly, the identification of fluid flow geometry and the determination of flow patterns in vertical wells are subject to the same problems.

Therefore, how to provide a method for measuring downhole fluid holdup that offers good versatility, high measurement efficiency, and low measurement cost, how to determine the fluid flow interface of a horizontal well, and how to determine the geometric distribution of each fluid phase of a vertical well are all technical problems that urgently need to be solved at present.

### SUMMARY

In view of the above technical problems, the present disclosure provides a method for measuring downhole holdup, and a method for determining downhole fluid interface and geometric distribution. The method for measuring downhole holdup uses multiple tuning forks distributed on a flow cross-section to achieve downhole fluid holdup measurement, offering advantages such as good versatility, high measurement efficiency, and high resolution. The method for determining a downhole fluid interface overcomes the technical difficulty of determining the oil-water interface in horizontal wells, providing a direct and effective approach for fluid interface determination in horizontal wells. The method for determining a downhole fluid geometric distribution overcomes the technical difficulty of intuitively displaying the fluid geometric distribution in vertical wells, providing a direct and effective approach for displaying fluid geometric distribution in vertical wells, which facilitates the determination of flow patterns in vertical wells.

A first aspect of the present disclosure provides a method for measuring a downhole holdup, employing a distributed tuning fork downhole fluid holdup measuring instrument for a measurement. The distributed tuning fork downhole fluid holdup measuring instrument includes a housing and at least eight tuning forks arranged at equal phase intervals along a circumferential direction of the housing, each tuning fork is connected to the housing via a corresponding telescopic arm, the telescopic arms corresponding to the tuning forks have different lengths to make multiple tuning forks distributed in a multi-turn ring shape relative to the axis of the housing after the telescopic arm is deployed.

The method for measuring includes the following steps:
S11, placing the distributed tuning fork downhole fluid holdup measuring instrument with the telescopic arms in a retracted state downhole along a wellbore, and deploying the telescopic arms so that multiple tuning forks are located at different positions of a flow cross-section to be measured downhole;
S12, using each tuning fork of the distributed tuning fork downhole fluid holdup measuring instrument to continuously collect a fluid density flowing through the position of the tuning fork, and when the fluid density collected by the tuning fork is greater than a preset oil-water density discrimination threshold, determining that the fluid currently flowing through the position of the tuning fork is water, otherwise determining it to be oil;
S13, according to formulas (1-1) and (1-2), calculating a local water holdup and a local oil holdup at the position of each tuning fork, respectively, and according to formulas (1-3) and (1-4), calculating a total water holdup and a total oil holdup corresponding to the flow cross-section to be measured, respectively;

$${y}_{w ( i )} = \frac{Σ {t}_{w ( i )}}{Σ {t}_{w ( i )} + Σ {t}_{o ( i )}} \qquad formula ( 1 - 1 )$$

$${y}_{o ( i )} = \frac{Σ {t}_{o ( i )}}{Σ {t}_{w ( i )} + Σ {t}_{o ( i )}} \qquad formula ( 1 - 2 )$$

$${y}_{w} = \frac{{\sum }_{i = 1}^{n} {y}_{w ( i )}}{n} \qquad formula ( 1 - 3 )$$

$${y}_{o} = \frac{{\sum }_{i = 1}^{n} {y}_{o ( i )}}{n} \qquad formula ( 1 - 4 )$$

where i=1, 2, 3 . . . n; n is a number of tuning forks, dimensionless; y_{w(i)} is a local water holdup at the position of an i^{th} tuning fork, %; yₒ₍ᵢ₎ is a local oil holdup at the position of the i^{th} tuning fork, %; Σt_{w(i)} is a cumulative total time that the fluid density collected by the i^{th} tuning fork is greater than the preset oil-water density discrimination threshold, s; Σtₒ₍ᵢ₎ is a cumulative total time that the fluid density collected by the i^{th} tuning fork is less than or equal to the preset oil-water density discrimination threshold, s; y_{w} is a total water holdup of the flow cross-section to be measured, %; yₒ is a total oil holdup of the flow cross-section to be measured, %.

In some of these embodiments, in step S12, an intermediate value between a measured density of downhole crude oil and a measured density of formation water in a region of a well to be measured is taken as the preset oil-water density discrimination threshold.

In some of these embodiments, step S12 further includes converting a fluid density signal collected by the tuning fork into a binary pulse signal so as to record a determination result, the specific conversion steps are: when the fluid density collected by the tuning fork is greater than a preset oil-water density discrimination threshold, sending out a signal of 1 and continuing to a next collection; and when the fluid density collected by the tuning fork is less than or equal to the preset oil-water density discrimination threshold, sending out a signal of 0 and continuing to the next collection; in step S13, a sum of durations when the pulse signal is 1 is taken as Σt_{w(i)}, and a sum of durations when the pulse signal is 0 is taken as ρtₒ₍ᵢ₎.

A second aspect of the present disclosure provides a method for determining a downhole fluid interface, applied to a horizontal well, employing a distributed tuning fork downhole fluid holdup measuring instrument to determine a fluid interface of the horizontal well. The distributed tuning fork downhole fluid holdup measuring instrument includes a housing and at least eight tuning forks arranged at equal phase intervals along a circumferential direction of the housing, each tuning fork is connected to the housing via a corresponding telescopic arm, the telescopic arms corresponding to the tuning forks have different lengths to make multiple tuning forks distributed in a multi-turn ring shape relative to the axis of the housing after the telescopic arm is deployed.

A method for determining a downhole fluid interface includes the following steps:
S21, horizontally placing the distributed tuning fork downhole fluid holdup measuring instrument with the telescopic arms in a retracted state into a measurement position of the horizontal well along a wellbore, and deploying the telescopic arms so that multiple tuning forks are located at different positions of a flow cross-section at the current measurement position;
S22, using each tuning fork of the distributed tuning fork downhole fluid holdup measuring instrument to continuously collect a fluid density flowing through the position of the tuning fork, and when the fluid density collected by the tuning fork is greater than a preset oil-water density discrimination threshold, determining that the fluid currently flowing through the position of the tuning fork is water, otherwise determining it to be oil; in a preset collection time, when a fluid flowing through a position of a certain tuning fork is continuously and alternately switched between water and oil for many times, identifying the position of the tuning fork as an oil-water interface point of a flow cross-section at the current measurement position, and identifying at least three oil-water interface points on the flow cross-section at the current measurement position;
S23, retracting the telescopic arms and moving the distributed tuning fork downhole fluid holdup measuring instrument to a next adjacent measurement position along a horizontal direction, deploying the telescopic arms so that multiple tuning forks are located at different positions of the flow cross-section at the current measurement position, and repeating step S22;
S24, repeating step S23, determining the oil-water interface points on the flow cross-section at several different measurement positions in the horizontal well, connecting the oil-water interface points and performing a surface fitting to obtain the oil-water interface of the horizontal well.

In some of these embodiments, in step S22, an intermediate value between a measured density of crude oil and a measured density of formation water in the horizontal well region to be measured is taken as the preset oil-water density discrimination threshold.

In some of these embodiments, in step S22, a preset collection time is 2-8 s, and a number of alternate switching is at least three.

In some of these embodiments, step S22 further includes: rotating the housing at the current measurement position to change the position of each tuning fork on the flow cross-section, repeating the collection until the oil-water interface point is identified.

A third aspect of the present disclosure provides a method for determining a downhole fluid geometric distribution, applied to a vertical well, employing a distributed tuning fork downhole fluid holdup measuring instrument to determine a geometric distribution of the vertical well. The distributed tuning fork downhole fluid holdup measuring instrument includes a housing and at least eight tuning forks arranged at equal phase intervals along a circumferential direction of the housing, each tuning fork is connected to the housing via a corresponding telescopic arm, the telescopic arms corresponding to the tuning forks have different lengths to make multiple tuning forks distributed in a multi-turn ring shape relative to the axis of the housing after the telescopic arm is deployed.

A method for determining a downhole fluid geometric distribution includes the following steps:
S31, vertically placing the distributed tuning fork downhole fluid holdup measuring instrument with the telescopic arms in a retracted state into the vertical well along a wellbore, and deploying the telescopic arms so that multiple tuning forks are located at different positions of the flow cross-section to be measured of the vertical well;
S32, using each tuning fork of the distributed tuning fork downhole fluid holdup measuring instrument to continuously collect a fluid density flowing through the position of the tuning fork, and when the fluid density collected by the tuning fork is greater than a preset oil-water density discrimination threshold, determining that the fluid currently flowing through the position of the tuning fork is water, otherwise determining it to be oil;
S33, according to formula (2-1), calculating a local oil holdup at the position of each tuning fork;

$${y}_{o ( i )} = \frac{Σ {t}_{o ( i )}}{Σ {t}_{w ( i )} + Σ {t}_{o ( i )}} \qquad formula ( 2 - 1 )$$

where i=1, 2, 3 . . . n; n is a number of tuning forks, dimensionless; yₒ₍ᵢ₎ is a local oil holdup at the position of an i^{th} tuning fork, %; Σt_{w(i)} is a cumulative total time that the fluid density collected by the i^{th} tuning fork is greater than the preset oil-water density discrimination threshold, s; Σtₒ₍ᵢ₎ is a cumulative total time that the fluid density collected by the i^{th} tuning fork is less than or equal to the preset oil-water density discrimination threshold, s;
S34, drawing a circular area to represent a flow cross-section to be measured of the vertical well, determining a corresponding position of each tuning fork in the circular area according to the position of each tuning fork on the flow cross-section to be measured, and for all tuning forks with a local oil holdup not being zero, drawing a circular oil bubble at the corresponding position of the tuning fork in the circular area, so as to obtain a fluid geometric distribution diagram corresponding to the flow cross-section; wherein an area of the circular oil bubble at the position corresponding to each tuning fork in the circular area is calculated according to formula (2-2);

$${S}_{i} = \frac{{y}_{o ( i )}}{n} S \qquad formula ( 2 - 2 )$$

where Sᵢ is an area of the circular oil bubble at the position of the i^{th} tuning fork, and S is an area of the circular area.

In some of these implementations, in step S32, an intermediate value between a measured density of crude oil and a measured density of formation water in the vertical well region to be measured is taken as the preset oil-water density discrimination threshold.

In some of these embodiments, step S32 further includes converting a fluid density signal collected by the tuning fork into a binary pulse signal so as to record a determination result, the specific conversion steps are: when the fluid density collected by the tuning fork is greater than a preset oil-water density discrimination threshold, sending out a signal of 1 and continuing to a next collection; and when the fluid density collected by the tuning fork is less than or equal to the preset oil-water density discrimination threshold, sending out a signal of 0 and continuing to the next collection, and a total number of collection times is 3-5; in the step S33, a sum of durations when the pulse signal is 1 is taken as Σt_{w(i)}, and a sum of durations when the pulse signal is 0 is taken as Σtₒ₍ᵢ₎.

Compared with the prior art, the advantages and beneficial effects of the present disclosure are as follows:
1. The method for measuring downhole holdup provided by the present disclosure is not only a new breakthrough in downhole holdup measurement technology beyond the capacitance-based, radioactivity-based, and electrode-probe-based techniques that have been used domestically and internationally since the 1950s and 1960s, but also breaks through the limitation that conventional tuning forks have been used only for measuring downhole fluid density. For the first time, the tuning fork is used to measure downhole fluid holdup, and oil-water determination is achieved based on the fluid density collected by the tuning fork. Then, the local oil holdup/local water holdup at the position of the tuning fork is characterized by the time proportion of oil/water flow passing the tuning fork as collected by the tuning fork. Based on the local oil holdup and local water holdup, the total water holdup and total oil holdup corresponding to the flow cross-section to be measured are calculated, thereby achieving characterization of downhole fluid holdup.
2. The method for measuring downhole holdup provided by the present disclosure is a novel downhole fluid holdup measurement method distinct from existing capacitive water holdup meters, radioactive water holdup meters, and downhole imaging logging technologies. It is not limited by high water-cut conditions or oil film wrapping, which can be adapted to various downhole conditions and can be applied to both vertical wells and horizontal wells, offering good versatility and high resolution. Moreover, its measurement steps are simple, continuous measurement can be performed without the need for standing, and measurement efficiency is high. Meanwhile, it does not require a radioactive source, resulting in a low measurement cost. These advantages make it significantly more effective than existing downhole fluid holdup measurement methods, and it is of great significance for guiding oilfield exploitation and test analysis.
3. The method for measuring downhole holdup provided by the present disclosure can achieve measurement of downhole fluid density while measuring downhole fluid holdup, thereby effectively reducing the workload of downhole fluid analysis and measurement;
4. The method for determining a downhole fluid interface provided by the present disclosure achieves oil-water determination based on the fluid density collected by the tuning fork, and then identifies the oil-water interface points of the horizontal well according to the phenomenon of alternating switching between oil and water. The oil-water interface of the horizontal well is determined by obtaining multiple oil-water interface points, thereby breaking through the technical problem that the oil-water interface of the horizontal well is difficult to determine, and providing a direct and effective method for fluid interface determination in the horizontal well. This method can provide data support for downhole flow imaging and is of great significance for guiding oilfield exploitation.
5. The method for determining a downhole fluid geometric distribution provided by the present disclosure achieves oil-water determination based on the fluid density collected by the tuning fork, and then combines the calculation of local oil holdup at the position of the tuning fork with the position distribution of the tuning forks in the flow cross-section to be measured, the fluid geometric distribution diagram is drawn, thereby breaking through the technical problem that the geometric distribution of fluid in a vertical well is difficult to display intuitively, and providing a direct and effective method for displaying the geometric distribution of fluid in the vertical well. This method facilitates the determination of flow patterns in the vertical well and can provide data support for downhole flow imaging, which is of great significance for guiding oilfield exploitation.

### BRIEF DESCRIPTION OF THE DRAWINGS

FIG. 1 is a front view of a distributed tuning fork downhole fluid holdup measuring instrument with telescopic arms in a retracted state according to Embodiment 1 of the present disclosure;

FIG. 2 is a front view of a distributed tuning fork downhole fluid holdup measuring instrument with telescopic arms in a deployed state according to Embodiment 1 of the present disclosure;

FIG. 3 is a side view of a distributed tuning fork downhole fluid holdup measuring instrument with telescopic arms in a deployed state according to Embodiment 1 of the present disclosure;

FIG. 4 is a flow chart of a method for measuring a downhole holdup according to Embodiment 1 of the present disclosure;

FIG. 5 is a schematic view of a distributed tuning fork downhole fluid holdup measuring instrument lowered downhole through a wellbore for collection in a method for measuring a downhole holdup according to Embodiment 1 of the present disclosure;

FIG. 6 is a flow chart of a method for determining a downhole fluid interface according to Embodiment 2 of the present disclosure;

FIG. 7 is a schematic view of a distributed tuning fork downhole fluid holdup measuring instrument performs a collection in a horizontal well in a method for determining a downhole fluid interface according to Embodiment 2 of the present disclosure;

FIG. 8 is a flow chart of a method for determining a downhole fluid geometric distribution according to Embodiment 3 of the present disclosure;

FIG. 9 is a schematic view of a distributed tuning fork downhole fluid holdup measuring instrument performs a collection in a vertical well in a method for determining a downhole fluid geometric distribution according to Embodiment 3 of the present disclosure;

FIG. 10 is a schematic view of a fluid geometric distribution diagram drawn in a method for determining a downhole fluid geometric distribution according to Embodiment 3 of the present disclosure.

### REFERENCE NUMERALS IN FIGURES

100, a distributed tuning fork downhole fluid holdup measuring instrument; 200, a wellbore; 300, a horizontal well; 400, an oil phase; 500, a water phase; 600, a vertical well; 700, a circular area; 800, a circular oil bubble;
110, a housing; 111, a first housing segment; 112, a second housing segment; 113, a telescopic rod;
120, a telescopic arm; 121, a first connecting rod; 122, a second connecting rod; 123, a tuning fork mounting seat;
130, a tuning fork; 131, an outer-ring tuning fork; 132, an inner-ring tuning fork; 140, a centralizer.

### DETAILED DESCRIPTION

The following clearly and completely describes the technical solutions in embodiments of the present disclosure. Apparently, the described embodiments are only some, but not all, of the embodiments of the present disclosure. All other embodiments obtained by those of ordinary skill in the art based on the embodiments of the present disclosure, without involving any creative effort, shall fall within the scope of protection of the present disclosure.

### Embodiment 1

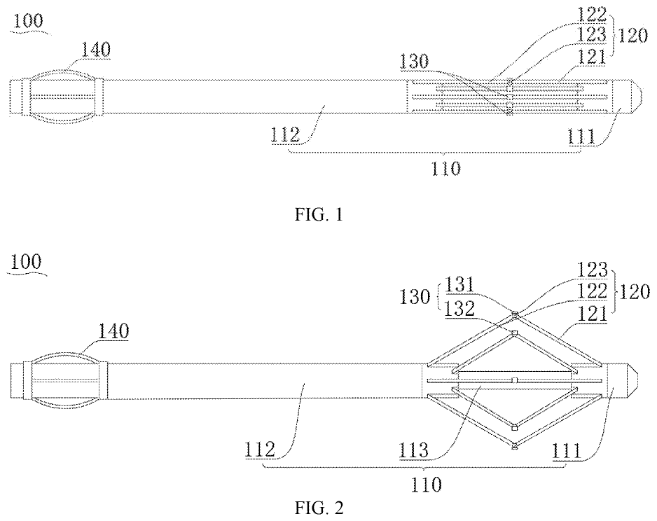

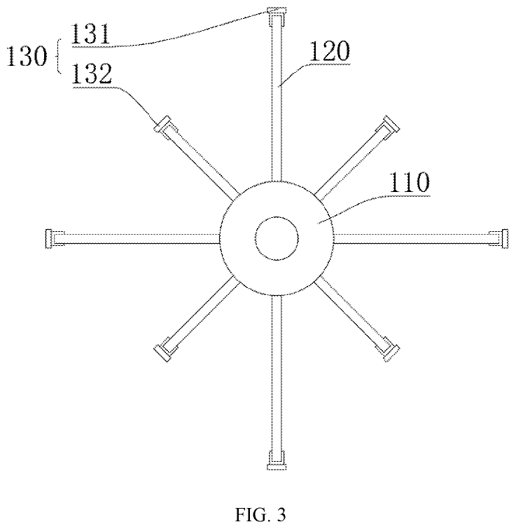

As shown in FIGS. 1-5, the embodiment provides a method for measuring a downhole holdup, employing a distributed tuning fork downhole fluid holdup measuring instrument 100 for measurement.

As shown in FIGS. 1-3, the distributed tuning fork downhole fluid holdup measuring instrument 100 includes a housing 110 and multiple tuning forks 130 arranged at equal phase intervals along a circumferential direction of the housing 110, wherein each tuning fork 130 is connected to the housing 110 via a corresponding telescopic arm 120; telescoping arm 120 has a retracted state abutting the outer wall of housing 110 and a deployed state extending relative to housing 110, with tuning fork 130 abutting the outer wall of housing 110 with telescoping arm 120 when telescoping arm 120 is in the retracted state, and with tuning fork 130 supported by telescoping arm 120 away from the outer wall of housing 110 when telescoping arm 120 is in the deployed state. With this arrangement, when the instrument is lowered, the telescopic arm 120 retracts to facilitate the instrument to pass through the wellbore 200. This prevents the tuning fork 130 from colliding with the wellbore 200 and causing damage. After arriving at the downhole measurement position, the telescopic arm 120 is deployed to enable multiple tuning forks 130 to be located at different positions of the downhole flow cross-section to be measured, thereby facilitating data collection at different positions of the downhole flow cross-section to be measured. It should be noted that the arrangement of multiple tuning forks 130 at equal phase intervals ensures a uniform distribution of the tuning forks over the flow cross-section to be measured. It should also be noted that, in order to improve the accuracy of the measurement results, the number of tuning forks 130 is at least eight, and a greater number of tuning forks 130 yields more accurate measurement results.

To achieve retraction and deployment of the telescopic arm 120, specifically, as shown in FIGS. 1-3, in the embodiment, the housing 110 includes a first housing segment 111 and a second housing segment 112 arranged coaxially. The first housing segment 111 is connected to the second housing segment 112 via a telescopic rod 113 so as to be capable of reciprocating relative to the second housing segment 112. An axial end of the first housing segment 111 away from the second housing segment 112 is an insertion end for extending into the downhole. The telescopic arm 120 includes a tuning fork mounting seat 123 for mounting the tuning fork 130, and a first connecting rod 121 and a second connecting rod 122 respectively hinged to two sides of the tuning fork mounting seat 123. The first connecting rod 121 and the second connecting rod 122 have the same length. One end of the first connecting rod 121 away from the tuning fork mounting seat 123 is hinged to an outer wall of the first housing segment 111, and one end of the second connecting rod 122 away from the tuning fork mounting seat 123 is hinged to an outer wall of the second housing segment 112. The telescopic rod 113 extends to drive the first housing segment 111 away from the second housing segment 112, and the first housing segment 111 drives the first connecting rod 121 and the second connecting rod 122 to rotate inwardly, thereby achieving retraction of the telescopic arm 120. The telescopic rod 113 retracts to drive the first housing segment 111 toward the second housing segment 112, and the first housing segment 111 drives the first connecting rod 121 and the second connecting rod 122 to rotate outwardly, thereby achieving deployment of the telescopic arm 120.

It should be noted that, as shown in FIGS. 1-3, after the telescopic arm 120 is deployed, multiple tuning forks 130 are distributed in a multi-turn ring shape relative to the axis of the housing 110. The length of the telescopic arm 120 (i.e., the total length of the first connecting rod 121 and the second connecting rod 122) connected by the tuning fork 130 located on the same turn is consistent, and the length of the telescopic arm 120 for an outer-ring tuning fork 131 is longer than the length of the telescopic arm 120 for an inner-ring tuning fork 132, so that when the telescopic rod 113 is retracted to drive the first housing segment 111 toward the second housing segment 112, the tuning forks 130 can be distributed in a multi-turn ring shape around the outer periphery of the housing 110.

It should also be noted that, as shown in FIGS. 1 and 2, in order to control the angle of the distributed tuning fork downhole fluid holdup measuring instrument 100, a centralizer 140 is also arranged on the outer periphery of the housing 110.

Additionally, a person skilled in the art could also use a distributed tuning fork downhole fluid holdup measuring instrument 100 having other structures, as long as the functions of “mounting the tuning fork 130 via the telescopic arm 120, and adjusting and controlling the position of the tuning fork 130 via retraction or deployment of the telescopic arm 120” are achieved. For example, based on the horizontal well water holdup measuring device disclosed in the existing China Patent CN210714671U, the tuning fork 130 may be used in place of the water holdup meter therein, wherein the tuning fork 130 employs an existing miniature tuning fork.

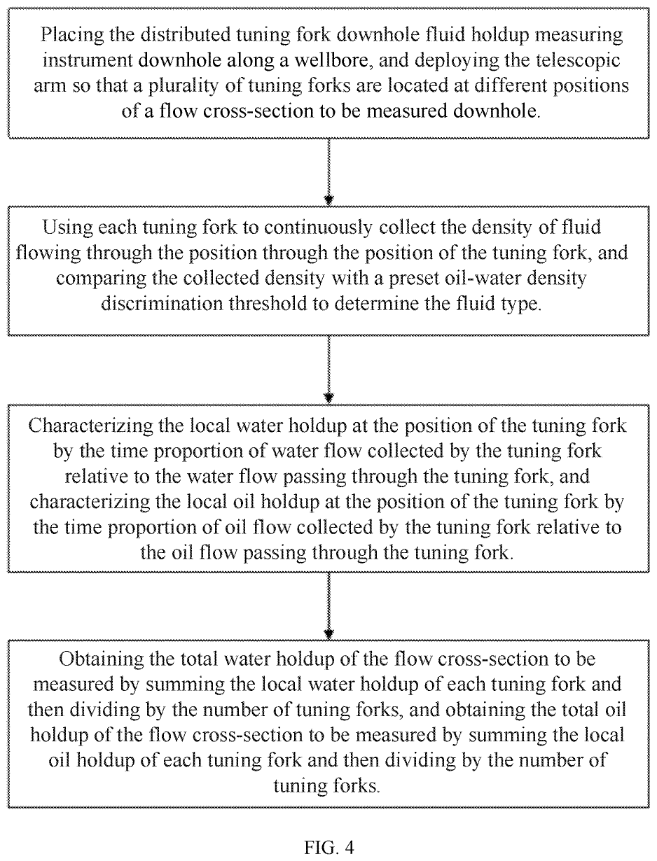

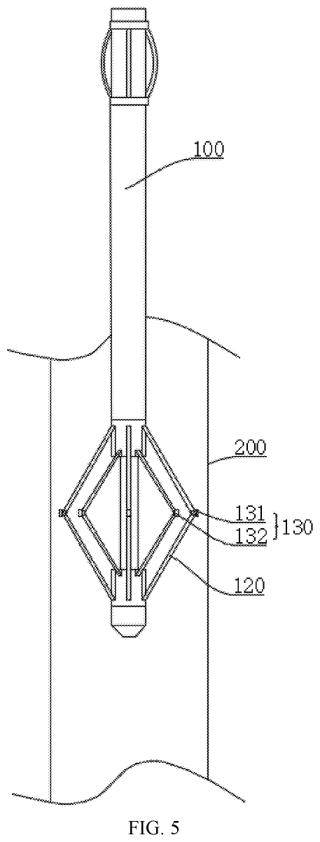

As shown in FIGS. 4 and 5, the method for measuring a downhole holdup provided by the embodiment includes the following steps:
S11, the distributed tuning fork downhole fluid capacity measuring instrument 100 with the telescopic arm 120 in a retracted state is placed downhole along the wellbore 200, and the telescopic arm 120 is deployed so that multiple tuning forks 130 are located at different positions of a flow cross-section to be measured downhole;

In this step, it should be noted that after the telescopic arm 120 is deployed, multiple tuning forks 130 are distributed in a multi-turn ring shape relative to the axis of the housing 110 on the flow cross-section to be measured.
S12, each tuning fork 130 of the distributed tuning fork downhole fluid holdup measuring instrument 100 is used to continuously collect the fluid density flowing through the position of the tuning fork 130, and when the fluid density collected by the tuning fork 130 is greater than the preset oil-water density discrimination threshold, determining that the fluid currently flowing through the position of the tuning fork 130 is water, otherwise determining it to be oil.

In this step, it should be noted that the following relationship exists between the fluid density flowing through the tuning fork and the vibration frequency of the tuning fork (i.e., the principle formula for density measurement using an existing tuning fork densitometer):

$$ρ = a + b {T}_{0} + c T$$

where ρ is the fluid density flowing through the tuning fork; T₀ is a natural frequency of the tuning fork; T is a frequency generated when the fluid flows through the tuning fork; and a, b, and c are constants that are related to the material, structure, and size of the tuning fork and can be obtained through experimental calibration.

There is a difference between the density of downhole crude oil (typically about 0.8 g/cm³) and the density of formation water (typically about 1.0 g/cm³). Therefore, in this step, based on the density difference between the oil and water phases, a preset oil-water density discrimination threshold is set. The tuning fork 130 is used to collect the fluid density flowing through the position of the tuning fork 130, and by comparing the fluid density collected by the tuning fork 130 with the preset oil-water density discrimination threshold, determination of the fluid type is achieved.

In some embodiments, the intermediate value between the measured density of downhole crude oil and the measured density of formation water in the region of the well to be measured is taken as the preset oil-water density discrimination threshold, which is conducive to ensuring the accuracy of fluid type determination.

Additionally, in order to simplify the amount of data and facilitate the subsequent calculation of the statistical time of fluid holdup, this step also includes converting the fluid density signal collected by the tuning fork 130 into the binary pulse signal so as to record the determination result, the specific conversion steps are: when the fluid density collected by the tuning fork 130 is greater than the preset oil-water density discrimination threshold, a signal of 1 is sent out and continued to the next collection; and when the fluid density collected by the tuning fork 130 is less than or equal to the preset oil-water density discrimination threshold, a signal of 0 is sent out and continued to the next collection.
S13, according to formulas (1-1) and (1-2), the local water holdup and the local oil holdup at the position of each tuning fork are calculated, respectively, and according to formulas (1-3) and (1-4), the total water holdup and the total oil holdup corresponding to the flow cross-section to be measured are calculated, respectively;

$${y}_{w ( i )} = \frac{Σ {t}_{w ( i )}}{Σ {t}_{w ( i )} + Σ {t}_{o ( i )}} \qquad formula ( 1 - 1 )$$

$${y}_{o ( i )} = \frac{Σ {t}_{o ( i )}}{Σ {t}_{w ( i )} + Σ {t}_{o ( i )}} \qquad formula ( 1 - 2 )$$

$${y}_{w} = \frac{{\sum }_{i = 1}^{n} {y}_{w ( i )}}{n} \qquad formula ( 1 - 3 )$$

$${y}_{o} = \frac{{\sum }_{i = 1}^{n} {y}_{o ( i )}}{n} \qquad formula ( 1 - 4 )$$

where i=1, 2, 3 . . . n; n is a number of tuning forks 130, dimensionless; y_{w(i)} is the local water holdup of the i^{th} tuning fork 130, %; yₒ₍ᵢ₎ is the local oil holdup at the position of the i^{th} tuning fork 130, %; Σt_{w(i)} is the cumulative total time that the fluid density collected by the i^{th} tuning fork 130 is greater than the preset oil-water density discrimination threshold, s; Σtₒ₍ᵢ₎ is the cumulative total time that the fluid density collected by the i^{th} tuning fork 130 is less than or equal to the preset oil-water density discrimination threshold, s; y_{w} is the total water holdup of the flow cross-section to be measured, %; yₒ is the total oil holdup of the flow cross-section to be measured, %.

Where the sum of durations when the pulse signal is 1 is taken as Σt_{w(i)}, and the sum of durations when the pulse signal is 0 is taken as Σtₒ₍ᵢ₎.

In this step, the local water holdup at the position of the tuning fork 130 is characterized by the time proportion of water flow collected by the tuning fork 130 relative to the water flow passing through the tuning fork 130, and the local oil holdup at the position of the tuning fork 130 is characterized by the time proportion of oil flow collected by the tuning fork 130 relative to the oil flow passing through the tuning fork 130. Then, based on the local water holdup and the local oil holdup, the total water holdup and the total oil holdup corresponding to the flow cross-section to be measured are calculated, thereby achieving measurement of downhole fluid holdup.

In this step, it should be noted that, since multiple tuning forks 130 are arranged and the sum of the total water holdup and the total oil holdup equals 1, when calculating the total water holdup and the total oil holdup, the local water holdup and local oil holdup of each tuning fork 130 are summed and then divided by the number of tuning forks 130.

In this step, it should be noted that, in order to ensure measurement accuracy and reduce measurement error, the average value of results collected over 3 to 5 collection periods is used for calculation. Preferably, the collection period is 1 min.

The method for measuring downhole holdup provided by the embodiment is not only a new breakthrough in downhole holdup measurement technology beyond the capacitance-based, radioactivity-based, and electrode-probe-based techniques that have been used domestically and internationally since the 1950s and 1960s, but also breaks through the limitation that conventional tuning forks have been used only for measuring downhole fluid density. For the first time, the tuning fork 130 is used to measure downhole fluid holdup, and oil-water determination is achieved based on the fluid density collected by the tuning fork 130. Then, the local oil holdup/local water holdup at the position of the tuning fork 130 is characterized by the time proportion of oil/water flow passing the tuning fork 130 as collected by the tuning fork 130. Based on the local oil holdup and local water holdup, the total water holdup and total oil holdup corresponding to the flow cross-section to be measured are calculated, thereby achieving characterization of downhole fluid holdup. The method is a novel downhole fluid holdup measurement method distinct from existing capacitive water holdup meters, radioactive water holdup meters, and downhole imaging logging technologies. It is not limited by high water-cut conditions or oil film wrapping, which can be adapted to various downhole conditions and can be applied to both vertical wells and horizontal wells, offering good versatility and high resolution. Moreover, its measurement steps are simple, continuous measurement can be performed without the need for standing, and measurement efficiency is high. Meanwhile, it does not require a radioactive source, resulting in a low measurement cost. These advantages make it significantly more effective than existing downhole fluid holdup measurement methods, and it is of great significance for guiding oilfield exploitation and test analysis. Additionally, the method for measuring downhole holdup provided by the embodiment can achieve measurement of downhole fluid density while measuring downhole fluid holdup, thereby effectively reducing the workload of downhole fluid analysis and measurement.

### Embodiment 2

This embodiment provides a method for determining a downhole fluid interface, applied to a horizontal well 300. The fluid interface of the horizontal well 300 is determined using the distributed tuning fork downhole fluid holdup measuring instrument 100. It should be noted that the structure of the distributed tuning fork downhole fluid holdup measuring instrument 100 used in this embodiment is the same as that of Embodiment 1, and thus is not repeated here.

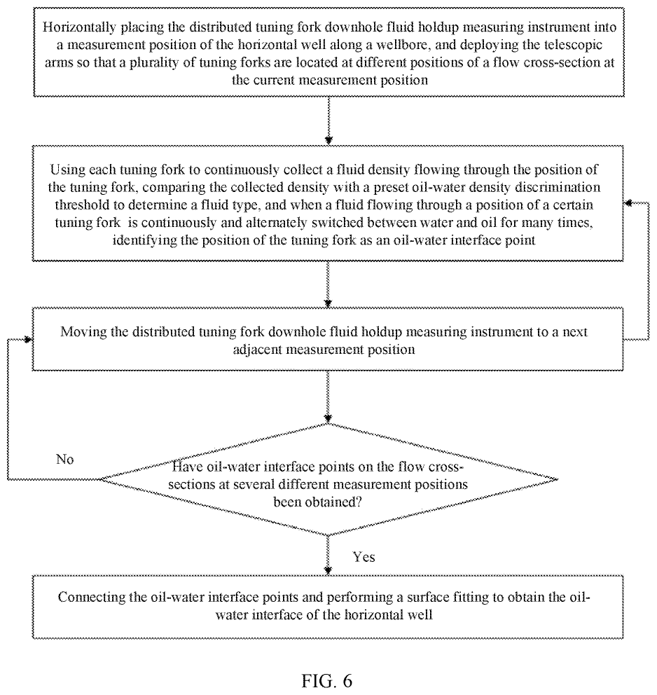

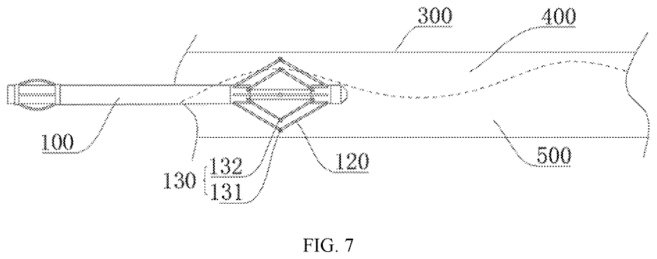

As shown in FIGS. 6 and 7, the method for determining a downhole fluid interface provided by the embodiment includes the following steps:
S21, the distributed tuning fork downhole fluid holdup measuring instrument 100 with the telescopic arms 120 in a retracted state is horizontally placed into a measurement position of the horizontal well 300 along a wellbore 200, and the telescopic arms 120 are deployed so that multiple tuning forks 130 are located at different positions of a flow cross-section at the current measurement position.

In this step, it should be noted that after the telescopic arm 120 is deployed, the multiple tuning forks 130 are distributed in a multi-turn ring shape relative to the axis of the housing 110 on the flow cross-section.
S22, each tuning fork 130 of the distributed tuning fork downhole fluid holdup measuring instrument 100 is used to continuously collect the fluid density flowing through the position of the tuning fork 130, and when the fluid density collected by the tuning fork 130 is greater than the preset oil-water density discrimination threshold, determined that the fluid currently flowing through the position of the tuning fork is water, otherwise determining it to be oil; in the preset collection time, when the fluid flowing through a position of the tuning fork 130 is continuously and alternately switched between water and oil for many times, the position of the tuning fork 130 is identified as an oil-water interface point of the flow cross-section at the current measurement position, and at least three oil-water interface points are identified on the flow cross-section at the current measurement position.

It should be noted that the principle of fluid type determination based on the fluid density collected by the tuning fork 130 in this step is the same as that in Embodiment 1, and is not repeated here. It should also be noted that in this step, when determining the fluid type based on the fluid density collected by the tuning fork 130, the intermediate value between the measured density of crude oil and the measured density of formation water in the horizontal well 300 to be measured is taken as the preset oil-water density discrimination threshold.

In the horizontal well 300, because the oil-water interface typically fluctuates up and down with a small amplitude, in this step, the oil-water interface point is identified based on whether the fluid flowing through the tuning fork 130 exhibits a phenomenon of continuous oil-water alternating switching multiple times. Specifically, when the fluid flowing through the tuning fork 130 remains water during a preset collection time, the position of the tuning fork 130 can be determined as a water phase 500. When the fluid flowing through the tuning fork 130 remains oil during the preset collection time, the position of the tuning fork 130 can be determined as an oil phase 400. When the fluid flowing through the tuning fork 130 exhibits multiple consecutive oil-water alternating switching phenomena during the preset collection time, this may be caused by upward and downward fluctuations of the oil-water interface, and the position of the tuning fork 130 can be determined as an oil-water interface point. To improve the accuracy of oil-water interface point determination, preferably, the preset collection time is 2-8 s, and the number of alternating switching is at least 3.

It should be noted that, as shown in FIG. 7, since the multiple tuning forks 130 are located at different positions of the flow cross-section at the current measurement position, multiple different positions of the flow cross-section can be identified simultaneously. In this step, at least three oil-water interface points are identified on the flow cross-section at each measurement position. By connecting the identified multiple oil-water interface points, an oil-water interface line can be drawn on the flow cross-section at the current measurement position. Understandably, the greater the number of identified oil-water interface points, the more accurate the oil-water interface line will be.

To increase the number of identified oil-water interface points, and particularly to avoid the situation where no oil-water interface point can be identified at a fixed placement angle of the distributed tuning fork downhole fluid holdup measuring instrument 100, preferably, this step further includes: the housing 110 at the current measurement position is rotated to change the position of each tuning fork 130 on the flow cross-section, and the collection is repeated until multiple oil-water interface points are identified.

It should be noted that, to avoid disturbance of the fluid in the horizontal well 300 when moving the distributed tuning fork downhole fluid holdup measuring instrument 100 affects the crude oil-water interface, after moving the distributed tuning fork downhole fluid holdup measuring instrument 100 or rotating its housing 110, the oil-water interface point is identified after allowing the instrument to stand for a certain period of time (e.g., 3 s).
S23, the telescopic arm 120 is retracted and the distributed tuning fork downhole fluid holdup measuring instrument 100 is moved to a next adjacent measurement position along a horizontal direction, the telescopic arm 120 is deployed so that multiple tuning forks 130 are located at different positions of the flow cross-section at the current measurement position, and step S22 is repeated.
S24, step S23 is repeated, the oil-water interface points on the flow cross-section at several different measurement positions in the horizontal well 300 are determined, the oil-water interface points are connected and the surface fitting is performed to obtain the oil-water interface of the horizontal well 300.

In this step, by repeatedly moving the distributed tuning fork downhole fluid holdup measuring instrument 100, identification of oil-water interface points on the flow cross-section at several different measurement positions in the horizontal well 300 can be achieved, that is, the oil-water interface line can be drawn for the flow cross-section at each measurement position. Therefore, by connecting the oil-water interface points on the flow cross-sections at several different measurement positions and then performing the surface fitting, the oil-water interface of the horizontal well 300 can be obtained.

The method for determining a downhole fluid interface provided by the embodiment achieves oil-water determination based on the fluid density collected by the tuning fork 130, and then identifies oil-water interface points of the horizontal well 300 according to the phenomenon of alternating switching between oil and water. The oil-water interface of the horizontal well 300 is determined by obtaining multiple oil-water interface points, thereby breaking through the technical problem that the oil-water interface of the horizontal well is difficult to determine, and providing a direct and effective method for fluid interface determination in the horizontal well. This method can provide data support for downhole flow imaging and is of great significance for guiding oilfield exploitation.

### Embodiment 3

This embodiment provides a method for determining a downhole fluid geometric distribution, which is applied to a vertical well 600. The fluid geometric distribution of the vertical well 300 is determined using the distributed tuning fork downhole fluid holdup measuring instrument 100. It should be noted that the structure of the distributed tuning fork downhole fluid holdup measuring instrument 100 used in this embodiment is the same as that of Embodiment 1, and thus is not repeated here.

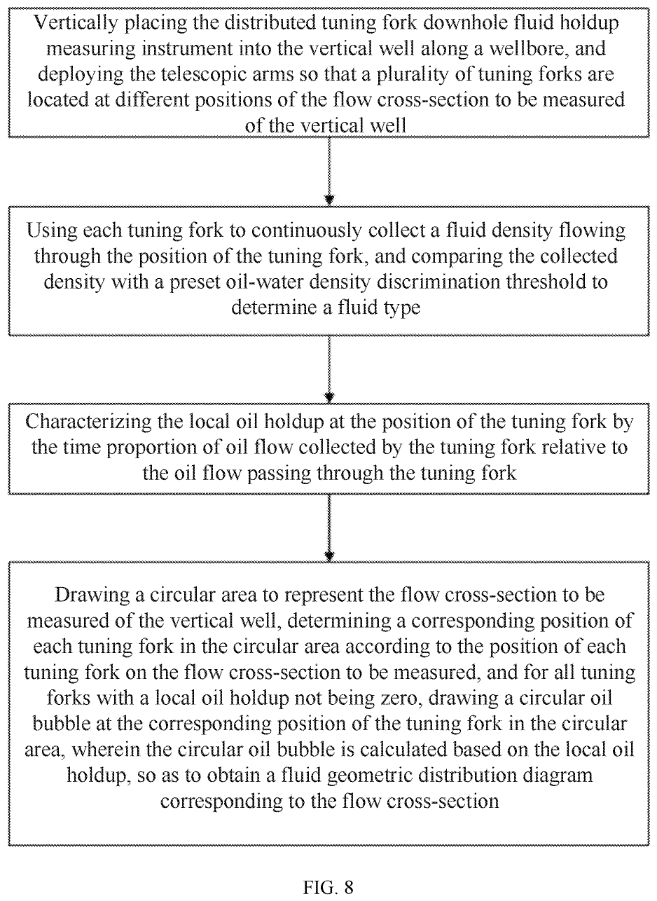

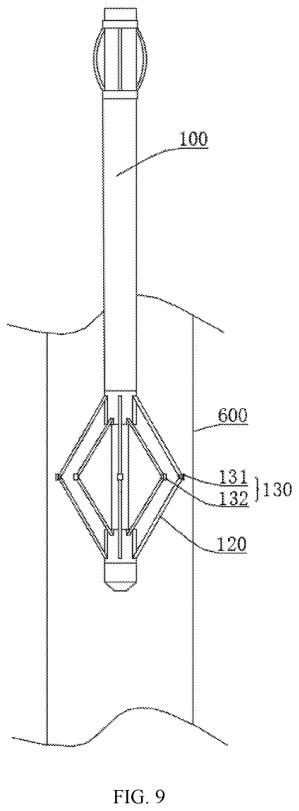

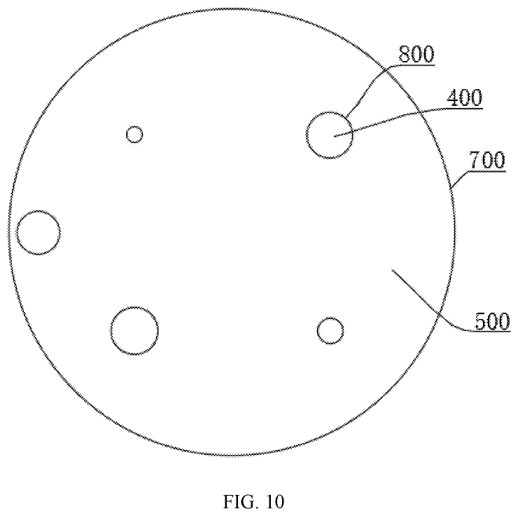

As shown in FIGS. 8 and 10, the method for determining a downhole fluid geometric distribution provided by the embodiment includes the following steps:
S31, the distributed tuning fork downhole fluid holdup measuring instrument 100 with the telescopic arms 120 in a retracted state is vertically placed into a measurement position of the vertical well 600 along a wellbore 200, and the telescopic arms 120 are deployed so that multiple tuning forks 130 are located at different positions of the flow cross-section to be measured of the vertical well 600.

In this step, it should be noted that after the telescopic arm 120 is deployed, the multiple tuning forks 130 are distributed in a multi-turn ring shape relative to the axis of the housing 110 on the flow cross-section.
S32, each tuning fork 130 of the distributed tuning fork downhole fluid holdup measuring instrument 100 is used to continuously collect the fluid density flowing through the position of the tuning fork 130, and when the fluid density collected by the tuning fork 130 is greater than the preset oil-water density discrimination threshold, determined that the fluid currently flowing through the position of the tuning fork is water, otherwise determining it to be oil.

It should be noted that the principle of fluid type determination based on the fluid density collected by the tuning fork 130 in this step is the same as that in Embodiment 1, and is not repeated here. It should also be noted that in this step, when determining the fluid type based on the fluid density collected by the tuning fork 130, the intermediate value between the measured density of crude oil and the measured density of formation water in the vertical well 600 to be measured is taken as the preset oil-water density discrimination threshold.

in order to simplify the amount of data and facilitate the subsequent calculation of the statistical time of fluid holdup, this step also includes converting the fluid density signal collected by the tuning fork 130 into the binary pulse signal so as to record the determination result, the specific conversion steps are: when the fluid density collected by the tuning fork 130 is greater than the preset oil-water density discrimination threshold, a signal of 1 is sent out and continued to the next collection; and when the fluid density collected by the tuning fork 130 is less than or equal to the preset oil-water density discrimination threshold, a signal of 0 is sent out and continued to the next collection, and a total number of collection times is 3-5. It should be noted that the fluid geometric distribution in the flow cross-section to be measured will change with time, and then the total number of collections may be controlled to 3-5 times.
S33, according to formula (2-1), the local oil holdup at the position of each tuning fork 130 is calculated;

$${y}_{o ( i )} = \frac{Σ {t}_{o ( i )}}{Σ {t}_{w ( i )} + Σ {t}_{o ( i )}} \qquad formula ( 2 - 1 )$$

where i=1, 2, 3 . . . n; n is the number of tuning forks 130, dimensionless; yₒ₍ᵢ₎ is the local oil holdup at the position of the i^{th} tuning fork 130, %; Σt_{w(i)} is the cumulative total time that the fluid density collected by the i^{th} tuning fork 130 is greater than the preset oil-water density discrimination threshold, s; Σtₒ₍ᵢ₎ is the cumulative total time that the fluid density collected by the i^{th} tuning fork 130 is less than or equal to the preset oil-water density discrimination threshold, s;

Wherein the sum of durations when the pulse signal is 1 is taken as Σt_{w(i)}, and the sum of durations when the pulse signal is 0 is taken as Σtₒ₍ᵢ₎.

It should be noted that the calculation principle of the local oil holdup at the position of each tuning fork 130 in this step is the same as that in Embodiment 1, which is not repeated here.
S34, the circular area 700 is drawn to represent the flow cross-section to be measured of the vertical well 600, the corresponding position of each tuning fork 130 in the circular area 700 is determined according to the position of each tuning fork 130 on the flow cross-section to be measured, and for all tuning forks 130 with the local oil holdup not being zero, the circular oil bubble 800 is drawn at the corresponding position of the tuning fork 130 in the circular area 700, so as to obtain the fluid geometric distribution diagram corresponding to the flow cross-section, wherein an area of the circular oil bubble 800 at the position corresponding to each tuning fork 130 in the circular area 700 is calculated according to formula (2-2);

$${S}_{i} = \frac{{y}_{o ( i )}}{n} S \qquad formula ( 2 - 2 )$$

where Sᵢ is the area of the circular oil bubble 800 at the position of the i^{th} tuning fork, and S is the area of the circular area 700.

In this step, it should be noted that when the local oil holdup at the position of the tuning fork 130 is not zero, the existence of the oil phase 400 at that position can be demonstrated. Furthermore, combined with the position of the tuning fork 130 on the flow cross-section to be measured, the circular oil bubble 800 can be drawn at the corresponding position within the circular area 700 representing the flow cross-section to be measured to represent the oil phase 400, and the area within the circular area 700 other than the circular oil bubble 800 represents the water phase 500. And the area ratio of the circular oil bubble 800 within the circular area 700 is calculated in combination with the local oil holdup. That is, the larger the local oil holdup, the more oil phase is present at that position, and thus the larger the area of the oil bubble. In this manner, the fluid geometric distribution diagram of the flow cross-section to be measured can be drawn.

The method for determining a downhole fluid geometric distribution provided by the embodiment achieves oil-water determination based on the fluid density collected by the tuning fork 130, and then combines the calculation of local oil holdup at the position of the tuning fork 130 with the position distribution of the tuning forks 130 in the flow cross-section to be measured, the fluid geometric distribution diagram is drawn, thereby breaking through the technical problem that the geometric distribution of fluid in the vertical well 600 is difficult to display intuitively, and providing a direct and effective method for displaying the geometric distribution of fluid in the vertical well 600. This method facilitates the determination of flow patterns in the vertical well 600 and can provide data support for downhole flow imaging, which is of great significance for guiding oilfield exploitation.

What is claimed is:

1. A method for measuring a downhole holdup, employing a distributed tuning fork downhole fluid holdup measuring instrument for a measurement, wherein the distributed tuning fork downhole fluid holdup measuring instrument comprises a housing and at least eight tuning forks arranged at equal phase intervals along a circumferential direction of the housing, each tuning fork is connected to the housing via a corresponding telescopic arm, the telescopic arms corresponding to the tuning forks have different lengths to make a plurality of tuning forks distributed in a multi-turn ring shape relative to the axis of the housing after the telescopic arm is deployed;
the method for measuring comprises the following steps:
S11, placing the distributed tuning fork downhole fluid holdup measuring instrument with the telescopic arms in a retracted state downhole along a wellbore, and deploying the telescopic arms so that a plurality of tuning forks are located at different positions of a flow cross-section to be measured downhole;
S12, using each tuning fork of the distributed tuning fork downhole fluid holdup measuring instrument to continuously collect a fluid density flowing through the position of the tuning fork, and when the fluid density collected by the tuning fork is greater than a preset oil-water density discrimination threshold, determining that the fluid currently flowing through the position of the tuning fork is water, otherwise determining it to be oil; and
S13, according to formulas (1-1) and (1-2), calculating a local water holdup and a local oil holdup at the position of each tuning fork, respectively, and according to formulas (1-3) and (1-4), calculating a total water holdup and a total oil holdup corresponding to the flow cross-section to be measured, respectively;
$${y}_{w ( i )} = \frac{Σ {t}_{w ( i )}}{Σ {t}_{w ( i )} + Σ {t}_{o ( i )}} \qquad formula ( 1 - 1 )$$
$${y}_{o ( i )} = \frac{Σ {t}_{o ( i )}}{Σ {t}_{w ( i )} + Σ {t}_{o ( i )}} \qquad formula ( 1 - 2 )$$
$${y}_{w} = \frac{{\sum }_{i = 1}^{n} {y}_{w ( i )}}{n} \qquad formula ( 1 - 3 )$$
$${y}_{o} = \frac{{\sum }_{i = 1}^{n} {y}_{o ( i )}}{n} \qquad formula ( 1 - 4 )$$
where i=1, 2, 3 . . . n; n is a number of tuning forks, dimensionless; y_{w(i)} is a local water holdup at the position of an i^{th} tuning fork, %; yₒ₍ᵢ₎ is a local oil holdup at the position of the i^{th} tuning fork, %; Σt_{w(i)} is a cumulative total time that the fluid density collected by the i^{th} tuning fork is greater than the preset oil-water density discrimination threshold, s; Σtₒ₍ᵢ₎ is a cumulative total time that the fluid density collected by the i^{th} tuning fork is less than or equal to the preset oil-water density discrimination threshold, s; y_{w} is a total water holdup of the flow cross-section to be measured, %; yₒ is a total oil holdup of the flow cross-section to be measured, %.

2. The method for measuring a downhole holdup according to claim 1, wherein in step S12, an intermediate value between a measured density of downhole crude oil and a measured density of formation water in a region of a well to be measured is taken as the preset oil-water density discrimination threshold.

3. The method for measuring a downhole holdup according to claim 1, wherein step S12 further comprises converting a fluid density signal collected by the tuning fork into a binary pulse signal so as to record a determination result, the specific conversion steps are: when the fluid density collected by the tuning fork is greater than the preset oil-water density discrimination threshold, sending out a signal of 1 and continuing to a next collection; and when the fluid density collected by the tuning fork is less than or equal to the preset oil-water density discrimination threshold, sending out a signal of 0 and continuing to the next collection; in step S13, a sum of durations when the pulse signal is 1 is taken as Σt_{w(i)}, and a sum of durations when the pulse signal is 0 is taken as Σtₒ₍ᵢ₎.

4. A method for determining a downhole fluid interface, applied to a horizontal well, employing a distributed tuning fork downhole fluid holdup measuring instrument to determine a fluid interface of the horizontal well, the distributed tuning fork downhole fluid holdup measuring instrument comprises a housing and at least eight tuning forks arranged at equal phase intervals along a circumferential direction of the housing, each tuning fork is connected to the housing via a corresponding telescopic arm, the telescopic arms corresponding to the tuning forks have different lengths to make a plurality of tuning forks distributed in a multi-turn ring shape relative to the axis of the housing after the telescopic arm is deployed;
the method for determining a downhole fluid interface comprises the following steps:
S21, horizontally placing the distributed tuning fork downhole fluid holdup measuring instrument with the telescopic arms in a retracted state into a measurement position of the horizontal well along a wellbore, and deploying the telescopic arms so that a plurality of tuning forks are located at different positions of a flow cross-section at the current measurement position;
S22, using each tuning fork of the distributed tuning fork downhole fluid holdup measuring instrument to continuously collect a fluid density flowing through the position of the tuning fork, and when the fluid density collected by the tuning fork is greater than a preset oil-water density discrimination threshold, determining that the fluid currently flowing through the position of the tuning fork is water, otherwise determining it to be oil; in a preset collection time, when a fluid flowing through a position of a certain tuning fork is continuously and alternately switched between water and oil for many times, identifying the position of the tuning fork as an oil-water interface point of a flow cross-section at a current measurement position, and identifying at least three oil-water interface points on the flow cross-section at the current measurement position;
S23, retracting the telescopic arms and moving the distributed tuning fork downhole fluid holdup measuring instrument to a next adjacent measurement position along a horizontal direction, deploying the telescopic arms so that a plurality of tuning forks are located at different positions of the flow cross-section at the current measurement position, and repeating step S22; and
S24, repeating step S23, determining the oil-water interface points on the flow cross-section at several different measurement positions in the horizontal well, connecting the oil-water interface points and performing a surface fitting to obtain the oil-water interface of the horizontal well.

5. The method for determining a downhole fluid interface according to claim 4, wherein in step S22, an intermediate value between a measured density of crude oil and a measured density of formation water in the horizontal well region to be measured is taken as the preset oil-water density discrimination threshold.

6. The method for determining a downhole fluid interface according to claim 4, wherein in step S22, a preset collection time is 2-8 s, and a number of alternate switching is at least three.

7. The method for determining a downhole fluid interface according to claim 4, wherein step S22 further comprises: rotating the housing at the current measurement position to change the position of each tuning fork on the flow cross-section, repeating the collection until the oil-water interface point is identified.

8. A method for determining a downhole fluid geometric distribution, applied to a vertical well, employing a distributed tuning fork downhole fluid holdup measuring instrument to determine a geometric distribution of the vertical well, the distributed tuning fork downhole fluid holdup measuring instrument comprises a housing and at least eight tuning forks arranged at equal phase intervals along a circumferential direction of the housing, each tuning fork is connected to the housing via a corresponding telescopic arm, the telescopic arms corresponding to the tuning forks have different lengths to make a plurality of tuning forks distributed in a multi-turn ring shape relative to the axis of the housing after the telescopic arm is deployed;
the method for determining a downhole fluid geometric distribution comprises the following steps:
S31, vertically placing the distributed tuning fork downhole fluid holdup measuring instrument with the telescopic arms in a retracted state into the vertical well along a wellbore, and deploying the telescopic arms so that a plurality of tuning forks are located at different positions of the flow cross-section to be measured of the vertical well;
S32, using each tuning fork of the distributed tuning fork downhole fluid holdup measuring instrument to continuously collect a fluid density flowing through the position of the tuning fork, and when the fluid density collected by the tuning fork is greater than a preset oil-water density discrimination threshold, determining that the fluid currently flowing through the position of the tuning fork is water, otherwise determining it to be oil;
S33, according to formula (2-1), calculating a local oil holdup at the position of each tuning fork;
$${y}_{o ( i )} = \frac{Σ {t}_{o ( i )}}{Σ {t}_{w ( i )} + Σ {t}_{o ( i )}} \qquad formula ( 2 - 1 )$$
where i=1, 2, 3 . . . n; n is a number of tuning forks, dimensionless; yₒ₍ᵢ₎ is a local oil holdup at the position of an i^{th} tuning fork, %; Σt_{w(i)} is a cumulative total time that the fluid density collected by the i^{th} tuning fork is greater than the preset oil-water density discrimination threshold, s; Σtₒ₍ᵢ₎ is a cumulative total time that the fluid density collected by the i^{th} tuning fork is less than or equal to the preset oil-water density discrimination threshold, s;
S34, drawing a circular area to represent a flow cross-section to be measured of the vertical well, determining a corresponding position of each tuning fork in the circular area according to the position of each tuning fork on the flow cross-section to be measured, and for all tuning forks with a local oil holdup not being zero, drawing a circular oil bubble at the corresponding position of the tuning fork in the circular area, so as to obtain a fluid geometric distribution diagram corresponding to the flow cross-section; wherein an area of the circular oil bubble at the position corresponding to each tuning fork in the circular area is calculated according to formula (2-2);
$${S}_{i} = \frac{{y}_{o ( i )}}{n} S \qquad formula ( 2 - 2 )$$
where Sᵢ is an area of the circular oil bubble at the position of the i^{th} tuning fork, and S is an area of the circular area.

9. The method for determining a downhole fluid geometric distribution according to claim 8, wherein in step S32, an intermediate value between a measured density of crude oil and a measured density of formation water in the vertical well region to be measured is taken as the preset oil-water density discrimination threshold.

10. The method for determining a downhole fluid geometric distribution according to claim 8, wherein step S32 further comprises converting the fluid density signal collected by the tuning fork into a binary pulse signal so as to record a determination result, the specific conversion steps are: when the fluid density collected by the tuning fork is greater than the preset oil-water density discrimination threshold, sending out a signal of 1 and continuing to a next collection; and when the fluid density collected by the tuning fork is less than or equal to the preset oil-water density discrimination threshold, sending out a signal of 0 and continuing to the next collection, and a total number of the collection times is 3-5; in step S33, a sum of durations when the pulse signal is 1 is taken as Σt_{w(i)}, and a sum of durations when the pulse signal is 0 is taken as Σtₒ₍ᵢ₎.
